(19) United States
(12) Patent Application Publication
(10) Pub. No.: US 2005/0142130 A1
(43) Pub. Date: Jun. 30, 2005

(54) Use of angiotensin-(1-7) for preventing and/or reducing the formation of neointima

(76) Inventors: Antonius Jacobus Marinus Roks, TL Groningen (NL); Yigal-Martin Pinto, VH Groningen (NL); Robert Henk Henning, AJ Loppersum (NL); Wiekert Hendrikus van Gilst, CA Haren (NL)

Correspondence Address: TRASK BRITT, P.O. BOX 2550, SALT LAKE CITY, UT, 84110, US

(21) Appl. No.: 10/946,329

(22) Filed: Sep. 20, 2004

Related U.S. Application Data
(63) Continuation-in-part of application No. 10/189,809, filed on Jul. 3, 2002.
Application No. 10/189,809 is a continuation of application No. PCT/NL01/00005, filed on Jan. 4, 2001.

(51) Int. Cl. A61K 38/48

(52) U.S. Cl. 424/94.64; 424/423

(57) ABSTRACT

Described is a method for preventing and/or reducing the formation of neointima comprising delivering to cells of an individual angiotensin-(1-7) or a functional part, derivative and/or analogue thereof, wherein use is made of a delivery vehicle that includes means for releasing angiotensin-(1-7) or a functional part, derivative and/or analogue thereof. Also described is a delivery vehicle for preventing and/or reducing the formation of neointima, wherein the delivery vehicle comprises an implantable device which device includes means for releasing angiotensin-(1-7)or a functional part, derivative and/or analogue thereof.

### CROSS-REFERENCE TO RELATED APPLICATIONS

This application is a continuation-in-part of U.S. patent application Ser. No. 10/189,809, filed Jul. 3, 2002, pending, which claims the benefit of U.S. Provisional Patent Application Ser. No. 60/176,172, filed Jan. 13, 2000, the contents of both of which are incorporated by this reference.

### TECHNICAL FIELD

The present invention relates generally to biotechnology, and more particularly to methods for preventing and/or reducing the formation of neointima, the use of delivery vehicles to establish this, and delivery vehicles as such.

### BACKGROUND OF THE INVENTION

Hypertension and hypercholesterolemia are two of the main risk factors for human health in the Western world; these conditions can lead to atherosclerosis. Atherosclerosis may result in a number of severe cardiovascular diseases, like chronic heart failure, angina pectoris, claudicatio intermittens, or peripheral and myocardial ischemia. At least the early phases of atherosclerosis are characterized by endothelial dysfunction. Endothelial dysfunction causes coronary arterial constriction and plays a role in both hypertension and hypercholesterolemia. It is one of the first measurable steps in the cascade of reactions leading to atherosclerosis, even before macroscopic lesions are evident. Many therapies have been investigated to assess the possibility to reverse the endothelial dysfunction, and to stimulate the formation of new blood vessels (angiogenesis). Examples are cholesterol reduction and ACE-inhibition.

It has been suggested that oral L-arginine supplementation in the diet may be a therapeutic strategy to improve angiogenesis in patients with endothelial dysfunction.

It is well established that angiogenesis is mediated by a multitude of cytokines (like TNF-α and E-selectin) and angiogenic factors including bFGF (basic Fibroblast Growth Factor), VEGF (Vascular Endothelial Growth Factor), and TGF-β. Both bFGF and VEGF are key regulators of angiogenesis in adult tissues. They selectively stimulate proliferation of endothelial cells, starting with the binding of these growth factors to receptors present on the endothelial cell surface.

Nitric oxide (NO) has been shown to play a role in this process. NO, originally identified as endothelium-derived relaxing factor, is an important endothelial vasoactive factor.

While both NO and angiogenic factors like bFGF and VEGF play a key role in the endothelial functions, their precise mode of action is not known. On the one hand, levels of angiogenic factors like bFGF and VEGF are increased in patients suffering from endothelial dysfunction. On the other hand, the release of nitric oxide in vascular endothelial dysfunction is often reduced. This reduced release may cause constriction of the coronary arteries and thus contributes to heart disease. It is postulated that patients suffering from endothelial dysfunction could benefit from therapies to increase new collateral blood vessel formation and/or therapies to increase vasodilatation.

Many experimental gene therapies concentrate on the stimulation of angiogenesis in patients suffering from endothelial dysfunction through the addition of VEGF or bFGF. Though these experimental therapies may have some effect, the level of therapy-induced angiogenesis is low, leading to a slow, if at all, recovery or enhancement of blood flow. The induction of angiogenesis is considered to be particularly relevant for cardiac-related diseases. While for most tissue other than the heart reduced blood flow is severely debilitating, reduced blood flow in the heart muscle is life threatening.

Cardiac tissue contains roughly two compartments consisting of cardio-myocytes and non-myocytes, respectively. The cardio-myocytes are highly differentiated cells which have lost the ability to divide and can adapt only by enlargement, so-called hypertrophy. The non-myocyte compartment consists of cells like fibroblasts, macrophages, vascular smooth muscle cells, vascular endothelial cells, endocardial cells and of an extracellular matrix. Enlargement of the non-myocyte compartment can be achieved by cell division and matrix deposition.

Physiological enlargement during normal development and growth and in response to intense exercise is characterized by an equal increase in both compartments. As a result, total myocardial contractility is increased. In contrast, myocardial adaptation in response to pressure/volume overload or myocardial infarction characteristically disturbs normal myocardial architecture, resulting in a relative increase of extracellular matrix and a decrease in capillary density^{1,2}. The relative deficit of capillaries is, in turn, the trigger for development of ischemia, which leads to deterioration of cardiac function in the long term.

The RAS (Renin Angiotensin System) is being considered as one of the most important regulatory systems for cardiovascular homeostasis. It plays a central role in blood pressure regulation, and in growth processes in the vessel wall, as well as the myocardium^{3,4}. The key enzyme, the angiotensin converting enzyme (ACE), that is abundantly present on endothelial cells, activates Ang II and inactivates bradykinin (BK). Ang II, which is formed from Ang I by ACE, is a vasoconstrictor and growth stimulator when acting on the AT1 receptor, while BK is a potent vasodilator. BK is degraded by ACE through sequential removal of the dipeptides Phe-Arg and Ser-Pro from the C-terminal end of the decapeptide. In addition to their inhibitory effect on Ang II formation, accumulation (and potentiation) of endogenous BK may be another mechanism by which ACE inhibitors exert their effects⁵.

The beneficial effects of ACE inhibitors on hypertrophied myocardium have been described extensively in animal and human studies³. Treatment with ACE inhibitors not only reduces symptoms, but also improves survival in heart failure patients⁴. Ang II is a potent growth factor for myocytes, fibroblasts, and vascular smooth muscle cell (VSMC). On a cellular level, multiple mechanisms play a role. Next to oncogenes and cyclins⁶, interference with cell cycle-regulating homeobox genes may be important. Ang II promotes unwanted VSMC proliferation by down-regulation of cell cycle-arresting genes, such as the growth arrest homeobox (gax)⁷. In this context, it is interesting that gene transfer with gax reduces porcine in-stent restenosis⁸.

The effect of BK on cell proliferation is less well described. It has been suggested that BK reduces fibroblast and VSMC proliferation by a prostaglandin- and NO-dependent mechanism. Given all the above, therefore, it is not surprising that up-regulation of (cardiac) ACE activity as found after myocardial infarction contributes to unfavorable remodeling of the myocardium: cardiomyocyte hypertrophy, increased matrix, and relative deficit of neovascularization or angiogenesis.

Angiogenesis, sprouting of new capillaries from the pre-existing vascular network, rarely occurs in the heart under normal conditions. Ang II has been described as an angiogenic factor,^{9, 10} while, at the same time, ACE inhibitors also have been described to exert angiogenesis-promoting activity¹¹⁻¹⁴. Although this seems contradictory, it might be explained by the stimulating effect of Ang II on VSMC to produce and release VEGF (mediated by the AT₁ receptor), which is a potent angiogenic factor¹⁵.

As already mentioned, ACE inhibition interferes not only with Ang II formation but also with the breakdown of BK. Since BK stimulates angiogenesis through BK₁ receptors¹⁶ and Ang II inhibits angiogenesis through AT₂-receptor¹⁵-mediated inhibition of endothelial cell (EC) proliferation, both effects of ACE inhibition may be pro-angiogenic in itself. Interference with the RAS may, therefore, have a dual synergistic effect, reduction of hypertrophy and extracellular matrix formation on the one hand and stimulation of angiogenesis on the other hand.

### SUMMARY OF THE INVENTION

In the present invention, it has been found that RAS interference by Ang (1-7), a member of circulating angiotensin peptides, prevents heart failure, presumably due to a synergism between reducing specific growth processes like myocardial and vascular hypotrophy on the one hand, and by stimulating myocardial angiogenesis, on the other hand. It seems promising, therefore, to further identify specific components of the RAS with regard to these specific actions.

The present invention makes use of the notion that heptapeptide Ang-(1-7), a member of circulating angiotensin peptides, which levels seem to be increased after ACE inhibition, functions as an endogenous inhibitor of the RAS. We show that Ang-(1-7) antagonizes the vasoconstrictor effects of Ang I and II. It has been shown that Ang-(1-7) enhances bradykinin B₂ receptor-mediated vasodilatation, displays antihypertensive actions in rats, and inhibits cultured rat VSMC growth. Importantly, since Ang-(1-7) also causes cardiac NO release, application of Ang-(1-7) in a gene therapy setting results in improved perfusion of the heart muscle, both directly through vasodilatation and indirectly through stimulation of NO-mediated angiogenesis.

Animal and cell culture studies demonstrate that Aug-(1-7) inhibits ACE activity, antagonizes AT₁ receptors, enhances BK-induced vasodilatation, and stimulates NO release via an Ang-(1-7)receptor^{20-22, 23-25}. This leads to the concept that Ang-(1-7) is an endogenous counterplayer of the renin-angiotensin system through a wide variety of mechanisms²⁶. The present invention employs the properties of Ang-(1-7) to modulate local growth processes in order to restore the balance between the above-described compartments and normalize myocardial architecture, and to make comparisons to other known growth modulators such as NO and VEGF. For this purpose, newly developed gene transfer vectors are used to induce specific and localized overexpression of these modulator substances at the site of interest.

Recent advances in the development of drug-eluting stents have led to a reduction in restenosis rates after stent implantation. Stents coated with rapamycin and paclitaxel inhibit the persistent smooth muscle cell proliferation after stenting. Recently, however, some potential drawbacks of these stents have emerged. Paclitaxel-eluting stents show delayed re-endothelialization and rapamycin inhibits endothelial cell proliferation. Consequently, refinement of anti-restenotic therapies remains mandatory. Particularly, repair of the normal biology of the vessel wall, by means of re-endothelialization, to prevent restenosis deserves special attention.

It has now been found that the use of an angiotensin-(1-7) has also a direct effect on the formation of neointima.

Accordingly, disclosed is a method for preventing and/or reducing the formation of neointima, comprising delivery to cells of an individual angiotensin-(1-7) or a functional part, derivative and/or analogue thereof, wherein use is made of a delivery vehicle which comprises a means for releasing the angiotensin-(1-7) or a functional part, derivative and/or analogue thereof.

The present invention is particularly attractive for preventing and/or reducing the formation of neointima around implantable devices that have been implanted in an individual. Such implantable devices include stents, catheters, pumps for dialysis purposes, and balloons for performing percutaneous angioplasty, but particularly, stents.

The angiotensin-(1-7) or a functional part, derivative and/or analogue thereof, can thus be released and delivered to intima that surround the implantable device. Delivery can be done in a local manner or a systemic manner. In the former manner, the implantable device comprises a means for releasing the angiotensin-(1-7) or a functional part, derivative and/or analogue thereof. Suitable systemic ways of releasing and delivering an angiotensin-(1-7) or a functional part, derivative and/or analogue thereof, include administration via pills, tablets, capsules, injections, catheters, pumps, sprays, infusion bags, and enteral and parenteral nutrition.

In the context of the present invention, the cells of the individual include adult and/or progenitor cells.

In a preferred embodiment, use is made of a nucleic acid delivery vehicle and the means that allows the release of a nucleic acid comprising at least one sequence encoding angiotensin-(1-7) or a functional part, derivative and/or analogue thereof, and the delivery vehicle further comprises a nucleic acid delivery carrier.

For the present invention, a functional analogue of angiotensin-(1-7) is angiotensin-(1-9)/Ang-(1-9) or angiotensin-(3-7). Since Ang-(1-9), like Ang-(1-7), is an Ace inhibitor (Kokonen et al. Circulation 1997, 95:1455-1463), and since both angiotensins resensitize the Bradykinin receptor (Marcic et al. Hypertension, 1999, 33, 835-843), a functional part, derivative and/or analogue of Ang-(1-7) and/or Ang-(1-9) comprises the same cardiac hypertrophy-inhibiting and/or preventing activity combined with myocardial angiogenesis-stimulating activity in kind, not necessarily in amount. On the other hand, some biological functions of Ang-(1-7) may result from conversion to Ang-(3-7), the latter being the ultimate mediator of that particular (yet unidentified) function.

When angiotensin-(1-7) is referred to in the present invention, this reference includes a functional part, derivative and/or analogue of angiotensin 1-7. Angiotensin-(1-7) is effective since it has an intrinsic vasodilatating effect in coronary arteries. Moreover, Ang-(1-7) is an ACE inhibitor and an antagonist of the unfavorable AT, receptor. Furthermore, angiotensin-(1-7) stimulates the release of prostacycline, which inhibits vasoconstriction. In a preferred embodiment, the nucleic acid delivery vehicle further comprises at least one sequence encoding an additional angiogenesis-promoting factor. These may be suitably chosen from the group of VEGF, bFGF, angiopoietin-1, a nucleic acid encoding a protein capable of promoting nitric oxide production, and functional analogues or derivatives thereof. Surprisingly, it has been found that under certain circumstances, a synergistic effect is obtained in the enhancing and/or inducing angiogenic effect. The additional angiogenesis-promoting factors may be supplied by sequences provided by the nucleic acid delivery vehicle or provided in other ways. They may also be provided by transduced cells or cells in the vicinity of surrounding transduced cells. In a preferred embodiment, the expression of at least one of said sequences is regulated by a signal. Preferably, the signal is provided by the oxygen tension in a cell. Preferably, the oxygen tension signal is translated into a different expression by a hypoxia-inducible factor 1α promoter. Considering that RAS is activated in a number of cardiovascular afflictions, promoters of the gene coding for ACE and the genes coding for angiotensin receptors are also preferred. An advantage of such a promoter is that the transcription of an RAS-inhibitor (Angiotensin 1-7), is turned on upon activation of transcription of unfavorable RAS components. Such a mechanism enables a production of Angiotensin-(1-7) predominantly when there is a need for it, thus obviating, at least in part, other control mechanisms for targeting expression to relevant cells.

In another aspect of the invention, the nucleic acid delivery vehicle may further comprise a sequence encoding a herpes simplex virus thymidine kinase, thus providing an additional method of regulating the level of enhanced and/or induced angiogenesis. The level may, at least in part, be reduced through the addition of gancyclovir, killing not only, at least in part, the dividing cells in the newly forming vessel parts, but also killing, at least in part, transduced cells, thereby limiting the supply of nitric oxide and/or additional angiogenesis-promoting factors.

The nucleic acid delivery carrier may be any nucleic acid delivery carrier, such as a liposome or virus particle. In a preferred embodiment of the invention, the nucleic acid delivery carrier comprises a Semliki Forest virus (SFV) vector, an adenovirus vector or an adeno-associated virus vector preferably including at least essential parts of SFV DNA, adenovirus vector DNA and/or adeno-associated virus vector DNA. Preferably, a nucleic acid delivery vehicle has been provided with at least a partial tissue tropism for muscle cells. Preferably, a nucleic acid delivery vehicle has been, at least in part, deprived of a tissue tropism for liver cells. Preferably, the tissue tropism is provided or deprived, at least in part, through a tissue tropism-determining part of fiber protein of a subgroup B adenovirus. A preferred subgroup B adenovirus is adenovirus 16.

The present invention also relates to a delivery vehicle for preventing and/or reducing the formation of neointima, wherein the delivery vehicle comprises an implantable device, which device comprises a means for releasing an angiotensin-(1-7) or a functional part, derivative and/or analogue thereof. In this way, an angiotensin-(1-7) or a functional part, derivative and/or analogue thereof can be released and delivered locally to the tissue that surround the implantable device. Suitable implantable devices include stents, catheters, pumps for dialysis purposes, and balloons for performing percutaneous angioplasty.

Preferably, the means for releasing an angiotensin-(1-7) or a functional part, derivative and/or analogue thereof comprises a layer which is coated on the implantable device, which layer comprises the angiotensin-(1-7) or a functional part, derivative and/or analogue thereof.

Preferably, the implantable device comprises a stent.

In a preferred embodiment of the present invention, the implantable device is a stent. Hence, the present invention also relates to a stent that has been coated with a layer which comprises an angiotensin-(1-7) or a functional part, derivative and/or analogue thereof.

The present invention provides a method for preventing and/or reducing the formation of neointima comprising providing cells of an individual, preferably a mammal, more preferably a human, with a delivery vehicle according to the invention and culturing the cells, preferably in vivo, under conditions allowing expression of a protein capable of increasing nitric oxide production. In another aspect, the invention provides a method for, at least in part, reducing hypertrophy comprising providing cells of an individual, preferably a mammal, more preferably a human, with a nucleic acid delivery vehicle according to the invention and culturing the cells, preferably in vivo, under conditions allowing expression of a protein capable of increasing nitric oxide production. In another aspect, the invention provides a method for enhancing and/or inducing angiogenesis comprising providing cells of an individual, preferably a mammal, more preferably a human, with a nucleic acid delivery vehicle according to the invention and allowing the cells to be cultured under conditions allowing expression of a protein capable of increasing nitric oxide production. As has been mentioned above, the method may be a method for enhancing and/or inducing angiogenesis in a synergistic fashion with at least one additional angiogenesis-promoting factor or parts, derivatives or functional analogues thereof. Preferably, the enhancing and/or inducing angiogenesis effect is at least in part reversible. Preferably, the effect is at least in part reversed though an increase in the oxygen tension or through providing the cells with gancyclovir or a functional analogue thereof, or both.

In a preferred aspect of the invention, at least cells are transduced that under normal circumstances are not in direct contact with blood; the advantage being that in this way, the treatment promotes, at least in part, the localization of the effect. Preferably, the cells not in direct contact with the blood are muscle cells, preferably cardiac or skeletal muscle cells, more preferably smooth muscle cells. Highly preferred cells in this regard are located in the heart of an individual suffering from, or at risk: of suffering from, heart pressure overload and/or myocardial infarction. Alternatively, the cells can be cardiac or vascular progenitor cells, either cultured in vitro or present in the organism, that can be treated either with a nucleic acid expressing Ang-(1-7), a derivative peptide, or with the peptide itself. When feasible, a preferred means of providing cells with a nucleic acid delivery vehicle of the invention is a catheter, preferably an Infiltrator catheter (EP 97200330.5). In another preferred method for providing cells with a nucleic acid delivery vehicle of the invention, the cells are provided with the nucleic acid delivery vehicle through pericardial delivery, preferably by a so-called perducer. Pericardial delivery is preferred since it limits the delivery to the relevant organ. Moreover, pericardial delivery is preferred since it results in a more even improvement of cardiac architecture.

The present invention also relates to a method for preventing and/or reducing vascular wall hypertrophy comprising delivery to cells of an individual angiotensin-(1-7) or a functional part, derivative and/or analogue thereof, wherein use is made of a delivery vehicle which comprises a means for releasing the angiotensin-(1-7) or a functional part, derivative and/or analogue thereof. Any of the delivery devices as described hereinbefore can be used for this purpose.

### BRIEF DESCRIPTION OF THE DRAWINGS

FIG. 1. Photomicrographs of haematoxylin-eosin stained sections of stented rat abdominal aortas. Panels A. and B: Aorta from control rat (x 40 and x 400, respectively). Panels C. and D: Aorta from Ang-(1-7)-treated rat (x 40 and x 400, respectively).

FIGS. 2A and 2B. Effects of stenting and Ang-(1-7) treatment on endothelial-dependent (FIG. 2A) and endothelial-independent dilation (FIG. 2B). FIG. 2A: Concentration-response curve to metacholine of phenylephrine precontracted aortic rings. p=0.009 vs. sham and p=0.001 vs. Ang-(1-7) treatment. FIG. 2B: Dilation to sodium nitrite (10 mM) of phenylephrine precontracted aortic rings. P=1.00 for sham vs. control and Ang-(1-7). PE indicates phenylephrine.

### DETAILED DESCRIPTION OF THE INVENTION

The invention will now be elucidated by the following non-restrictive examples.

### EXAMPLES

Animal Protocol

Twenty-eight male Wistar rats (Harlan, Horst, Netherlands) weighing 450 to 520 grams were anesthetized with O₂, N₂O and isofluorane (Abbot B.V., Hoofddorp, Netherlands). A pre-mounted 2.5×9 mm BeStent™ 2 (Medtronic-Bakken Research, Maastricht, the Netherlands) was implanted in the abdominal aorta as previously described, or a sham operation was performed.³⁵ Subsequently, an osmotic minipump with a pumping rate of 0.25 μl/hour, lasting for 28 days (Model 2004, Alzet, Charles River Nederland, Maastricht, Netherlands), was implanted subcutaneously for drug delivery via a catheter in the jugular vein. Stented rats received angiotensin-(1-7) (Bachem, Weil am Rhein, Germany) (24 μg/kg/hour) (n=7) or saline (0.25 μl/hour) (n=10). Sham-operated rats received saline infusion (n=6). With this method, Ang-(1-7) plasma levels of approximately 917.8±194.1 pmol/l are reached. At this concentration, Ang-(1-7) binds to the Mas receptor and has subsequent functional effects. Five rats died peri-operatively due to rupture of the aorta.

After 28 days, the animals were anesthetized and heparinized with 500 IU intravenously (Leo Pharma B.V., Breda, Netherlands). The abdominal aortas were subsequently harvested, fixed, embedded in methylmetacrylate, sectioned and stained for histological analysis. The endothelial function was tested in isolated thoracic aortic rings.

These experiments were approved by the Animal Care and Use Committee of the University of Groningen and performed in accordance with the “Guide for the Care and Use of Laboratory Animals.”

Histology

Histomorphometrical analysis was performed on elastica van Gieson-stained sections by measurements of the proximal, middle and distal parts of each stent. To assess neointimal formation, areas within the external elastic lamina (EEL), internal elastic lamina (IEL) and lumen were measured by using digital morphometry. The neointimal area, media area, lumen area and the percentage of stenosis were calculated.

The injury and inflammation scores were assessed as described by Schwartz et al. and Kornowski et al. Briefly, each strut was assigned a nominal score from 0 to 3 dependent upon the severity of the injury or inflammation. The average score is calculated by dividing the sum of scores by the number of struts. Total cell density and polymorphonuclear leukocyte density were determined in hematoxylin-eosin stained sections at ×400 magnification and expressed as ×100/mm². To assess a single measurement for each stent, the mean values of the proximal, middle and distal parts were calculated.

Organ Bath Studies with Isolated Aortic Rings

Peri-aortic tissue was removed from the aorta and rings of approximately 2 mm were cut. The rings were connected to an isotonic displacement transducer at a preload of 14 nM in an organ bath containing Krebs solution (pH 7.5) containing (mM): NaCl (120.4), KCl (5.9), CaCl₂ (2.5) MgCl₂ (1.2), NaH₂PO₄ (1.2), glucose (11.5), NaHCO₃ (25.0), at 37° C. and continuously gassed with 95% O₂ and 5% CO₂. After stabilization, during which regular washing was performed, rings were checked for viability by stimulation with phenylephrine (1 mM).

The rings were washed and restabilized. Sets of rings were precontracted with phenylephrine (1 mM). The endothelium-dependant vasodilatation was assessed by a cumulative dose of meatcholine (10 nM to 10 mM). Subsequently, the rings were dilated maximally by means of the endothelium-independent vasodilator sodium nitrite (10 mM). Drugs were purchased from Sigma-Aldrich, Steinheim, Germany.

Statistics

Data are expressed as mean value±standard error of the mean (SEM). Statistical analysis between groups was performed by a student's t-test. Differences in dose-response curves between groups were tested by ANOVA for repeated measures using Greenhouse-Geisser correction for asphericity. Values of p=0.05 were considered statistically significant. For statistical analysis, SPSS software (Chicago, USA) was used.

Results

Histological Analysis

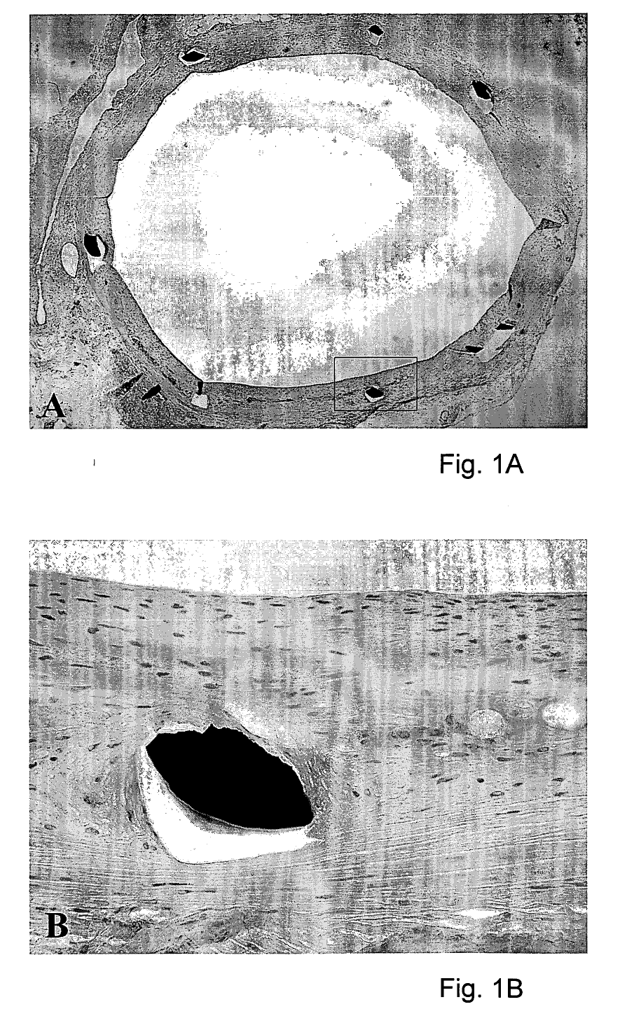

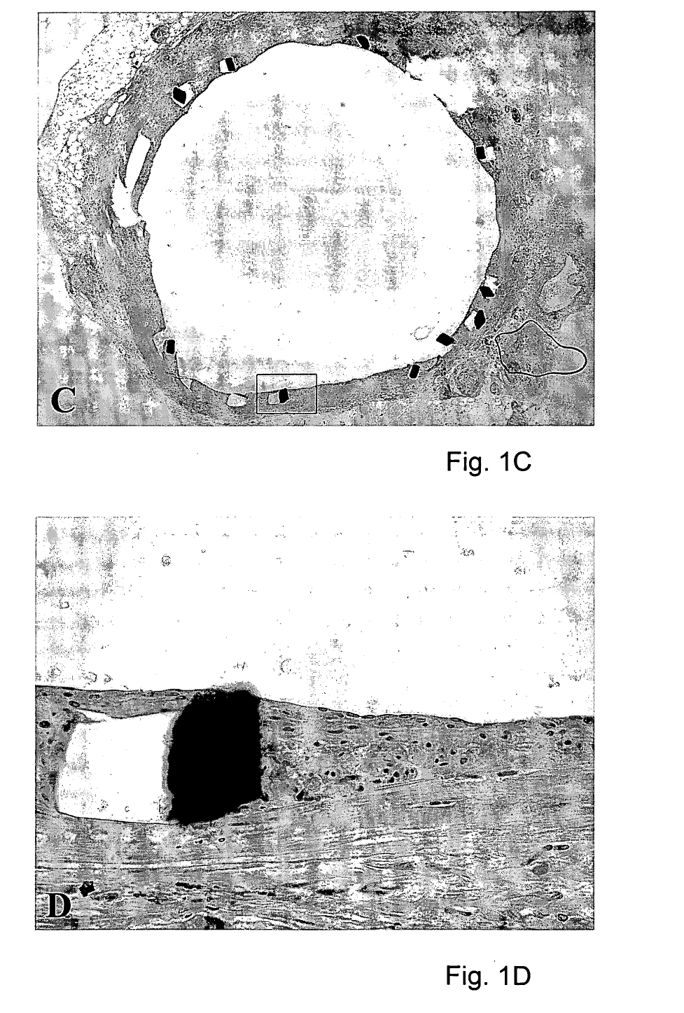

In all stented animals, a neointima was present after 28 days, on which histological analysis was performed. Histomorphometric measurements are presented in Table 1. Stent expansion, expressed as the IEL area, was equal in the saline- and the Ang-(1-7)-treated groups. Accordingly, the mean injury score also did not show a difference between the groups. Furthermore, no differences were observed in the media areas. Neointimal thickness, neointimal area and percentage stenosis were significantly decreased in the Ang-(1-7)-treated group, with 21%, 27% and 26%, respectively. Representative photomicrographs of stented abdominal aortas of the saline- and Ang-(1-7)-treated animals are shown in FIG. 1.

Histological measurements are presented in Table 2. The cellular density in the media of the Ang-(1-7)-treated group was diminished as compared to the control group. No difference was observed in the cellular density in the neointima. The number of surface-adherent leukocytes appeared to be decreased in the Ang-(1-7) group, almost reaching the level of significance (p=0.06). The neointimal density of polymorphonucloar leukocytes and the mean inflammation score, which represent the infiltrated inflammatory cells, did not differ between groups.

Endothelial Function

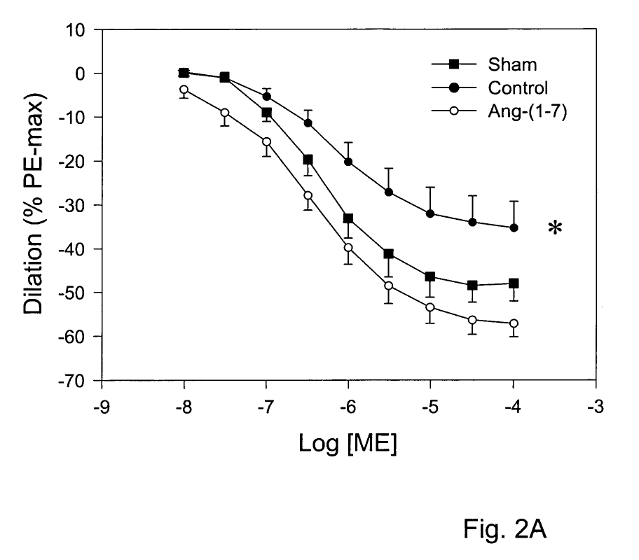

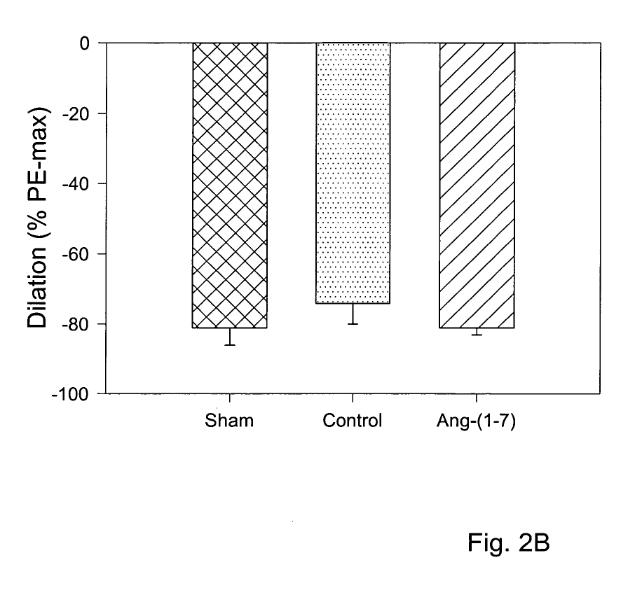

The effects of stent implantation in the rat abdominal aorta, and subsequent Ang-(1-7) infusion on endothelial function were examined in thoracic aortic rings. We investigated the endothelium-dependent vasodilatory effects of metacholine on phenylephrine precontracted rings (FIG. 2A). The contraction on phenylephrine was similar in the sham, control and Ang-(1-7) group (329±26, 297±20 and 254±29 μm, respectively. P=1.00 and p=0.20 for sham vs. control and Ang-(1-7), respectively). Stenting resulted in a significant decline of 13% in endothelium-dependent relaxation as compared to the sham-treated animals. In the Ang-(1-7)-treated group, we observed a significant improvement of 21% in vasodilatory response to metacholine as compared to the saline-treated group. The vasodilatory response in the Ang-(1-7) group seemed to exceed the response in the sham animals; however, this was not significant (p=0.952) (FIG. 2A). The relaxation on endothelium-independent vasodilator sodium nitrite was equal in the sham, control and Ang-(1-7) group (FIG. 2B).

Discussion

In the Examples, the effect of Ang-(1-7) infusion on neointimal formation in a rat stenting model is shown. A significant reduction in neointimal thickness, neointimal area and percentage stenosis after Ang-(1-7) treatment was observed of 21%, 27% and 26%, respectively. Additionally, it was found that an attenuation of the stent-induced impairment of endothelium-dependent relaxation after Ang-(1-7) administration. Ang-(1-7) treatment resulted in an improvement of 39% of endothelium-dependent relaxation in aortic rings. No differences in endothelial-independent relaxation were observed. These results indicate a strong improvement of endothelial function.

Restenosis after stent implantation ensues from focal thrombus formation, inflammation and smooth muscle cell proliferation after deep injury to the vessel wall and deendothelialization. Thrombus formation and smooth muscle cell proliferation are diminished by Ang-(1-7). Moreover, Ang-(1-7) infusion reduces neointimal formation and smooth muscle cell proliferation after vascular injury in the rat carotid artery. Ang-(1-7) inhibits neointimal formation after stenting.

These results show that Ang-(1-7) treatment after stent implantation in the rat abdominal aorta results in attenuation of neointimal formation, combined with an improvement of endothelial function. Ang-(1-7) may be an important alternative to the presently available aggressive anti-proliferative drug-eluting stents.

TABLE 1
Histomorphometric measurements

| | | | Change | |
|---|---|---|---|---|
| | | | with | |
| | | Ang-(1-7) | treatment | |
| | Control | infusion | (%) | P-value |
| Mean Injury Score | 0.93 ± 0.07 | 1.10 ± 0.16 | 18.2 | 0.357 |
| IEL Area (mm²) | 5.03 ± 0.15 | 4.92 ± 0.32 | −2 | 0.774 |
| Media Area (mm²) | 0.47 ± 0.04 | 0.41 ± 0.05 | −12.8 | 0.314 |
| Neointimal | 141 ± 11 | 112 ± 8 | −20.6 | 0.046 |
| Thickness (μm) | | | | |
| Neointimal Area | 0.70 ± 0.07 | 0.51 ± 0.05 | −27.1 | 0.038 |
| (mm²) | | | | |
| Percentage | 14.0 ± 1.3 | 10.4 ± 1.0 | −25.7 | 0.050 |
| Stenosis (%) | | | | |

IEL indicates internal elastic lamina.

TABLE 2
Histological measurements

| | | Ang-(1-7) | |
|---|---|---|---|
| | Control | infusion | P-Value |
| Media Cell Density | 11.21 ± 1.17 | 6.93 ± 1.37 | 0.036 |
| (×100/mm²) | | | |
| Intima Cell Density | 47.53 ± 2.57 | 52.64 ± 6.89 | 0.511 |
| (×100/mm²) | | | |
| Polymorphonuclear | 0.28 ± 0.16 | 0.19 ± 0.09 | 0.644 |
| Leukocytes (×100/mm²) | | | |
| Surface Adherent | 5.6 ± 1.1 | 2.8 ± 0.8 | 0.061 |
| Leukocytes (cells/section) | | | |
| Mean Inflammation Score | 0.32 ± 0.03 | 0.32 ± 0.08 | 0.992 |

### References

1. Weber K. T., Brilla C. G. Pathological hypertrophy and cardiac interstitium, fibrosis and renin-angiotensin-aldosteron system. Circulation 1991; 83:1849-1865.
2. Weber K. T. Cardiac interstitium in health and disease: the fibrillar collagen network. J. Am. Coll. Cardiol. 1989; 13:1637-1652.
3. Brown N. J., Vaughan D. E. Angiotensin-converting enzyme inhibitors. Circulation 1998; 97:1411-1420.
4. Chryssant S. G. Vascular remodeling: The role of angiotensin-converting enzyme inhibitors. Am. Heart J. 1998; 135 (Spt2):S21-S30.
5. Campbell D. J., Kladis A., Duncan A.-M. Effects of converting enzyme inhibitors on angiotensin and bradykinin peptides. Hypertension 1994; 23:439-449.
6. Diez J., Panizo A., Hernandez M., Galindo M. E., Cenarruzabeitir E., Parodo Mindan F. J. Quinapril inhibits c-Myc expression and normalizes smooth muscle cell proliferation in spontaneous hypertensive rats. Am. J. Hypertens. 1997; 10 (Pt2):1147-1152.
7. Yamashita J., Itoh H., Ogawa Y., Tamura N., Takaya K., Igaki T., Doi K., Chun T. H., Inoue M., Masatsugu K., Nakao K. Opposite regulation of Gax homeobox expression by angiotensin II and C-type natriuretic peptide. Hypertension 1997 (1Pt2): 381-387.
8. Tio R. A., Smith R. C., Scheuermann T. H., Lebherz C., Luo Z., Tkebuchava T., Duverger N., Branellec D., Isner J. M., Walsh K. Adenovirus-mediated Gax gene therapy in stented porcine coronary arteries: delivery, dissemination and efficacy (submitted).
9. Amant C., Bauters C., Bodart J. C., Lablanche J. M., Grollier G., Danchin N., Hamon M., Richard F., Helbecque N., McFadden E., Amouyel P., Bertrand M. D. Allele of the angiotensin I-converting enzyme is a major risk factor for restenosis after coronary stenting. Circulation 1997; 96:56-60.
10. Fernandez L. A., Twickler J., Mead A. Neovascularization produced by angiotensin II. J. Lab. Clin. Med. 1985; 105:141-145.
11. leNoble F. A. C., Schreurs N. H. J. S., van Straaten H. W. M., Slaaf J. F. M., Rogg H., Struijker-Boudier A. J. Evidence for a novel angiotensin II receptor involved in angiogenesis in chick chorioallantoic membrane. Am. J. Physiol. 1993; 264:R460-R465.
12. Olivetti G., Cigola E., Lagrasta C., Ricci R., Quaini F., Mnopoli A., Ongini E. Spirapril prevents left ventricular hypertrophy, decreases myocardial damage and promotes angiogenesis in spontaneously hypertensive rats. J. Cardiovasc. Pharmacol. 1993; 21:362-370.
13. Unger T., Mattfeldt T., Lamberty V., Bock P., Mall G., Linz W., Scholkens B. W., Gohlke P. Effects of early onset angiotensin-converting enzyme inhibition on myocardial capillaries. J. Hypert. 1992; 20:487-482.
14. Cameron N. E., Cotter M. A., Robertson S. Angiogtensin-converting enzyme inhibition prevents the development of the muscle and nerve dysfunction and stimulates the angiogenesis in streptozotocin-diabetic rats. Diabetologia 1992; 35:12-18.
15. Stoll M., Meffert B., Stroth U., Unger T. Growth or antigrowth: angiotensin and the endothelium. J. Hypert. 1995; 13:1529-1534.
16. Hu D. E., Fran T. P. D. [Leu⁸]des-Arg⁹-bradykinin inhibits the angiogenic effects of bradykinin and interleukin-1 in rats. Br. J. Pharmacol. 1993; 109:14-17.
17. Voors A. A., Pinto Y. M., Buikema H., Utara H., Rooks G., Grandjean J. G., Ganten D., van Gilst W. H. Dual pathway for angiotensin II formation in human internal mammary arteries. Br. J. Pharmacol. (accepted).
18. Roks A. J. M., Buikema H., Pinto Y. M., van Gilst W. H. The renin-angiotensin system and vascular function. The role of angiotensin II, angiotensin-converting enzyme, and alternative conversion of angiotensin I. Heart Vessels 1997; S12:119-124.
19. Nishimura H., Buikema H., Baltatu O., Ganten D., Urata H. Functional evidence for alternative angiotensin II-forming pathways in hamster cardiovascular system. Am. J. Physiol (accepted).
20. Paula R. D., Lima C. V., Khosla M. C., Santos R. A. S. Angiotensin-(1-7) potentiates the hypotensive effects of bradykinin in conscious rats. Hypertension 1995; 26:1154-1159.
21. Deddish P. A., Marcic B., Jackman H. L., Wang H.-Z., Skidgel R. A., Erdös E. G. N-domain-specific substrate and C-domain inhibitors of angiotensin-converting enzyme. Angiotensin-(1-7) and Keto-ACE. Hypertension 1998; 31:912-917.
22. Brosnihan K. B., Li P., Ferraro C. M. Angiotensin-(1-7) dilates canine coronary arteries through kinins and nitric oxide. Hypertension 1996; 27:523-528.
23. Mahon J. M., Carr R. D., Nicol A. K., Henderson I. W. Angiotensin-(1-7) is an antagonist at the type I angiotensin II receptor. J. Hypertens. 1994; 12:1377-1381.
24. Pörsti I., Bara A., Busse R., Hecker M. Release of nitric oxide by angiotensin-(1-7) from porcine coronary endothelium: implications for a novel angiotensin receptor. Br. J. Pharmacol. 1994; 111 :625-654.
25. Tallant E. A., Lu X., Weiss R. B., Chapell M. C., Ferario C. M. Bovine aortic endothelial cells contain an angiotensin-(1-7) receptor. Hypertension 1997; 29(2):388-398.
26. Ferario C. M., Chapell M. C., Tallant A., Brosnihan B., Diz D. I. Counterregulatory actions of angiotensin-(1-7). Hypertension 1997; 30(2):585-541.
27. Yamamoto K., Chappell M. C., Brosnihan K. B., Ferrario C. M. In vivo metabolism of angiotensin I by neutral endopeptidase (EC 3.4.24.11) in spontaneously hypertensive rats. Hypertension 1992; 19:692-696.
28. Berglund P, Sjöberg M., Garoff H., Atkins G. J., Sheahan B. J., Liljestrom P. Semliki Forest virus expression system: production of conditionally infectious recombinant particles. Bio/technol. 1993; 11:916-920.

1. A method for preventing and/or reducing the formation of neointima in an individual, said method comprising:
delivering, to cells of an individual, angiotensin-(1-7) or a functional part, derivative and/or analogue thereof,
wherein use is made of a delivery vehicle comprising means for releasing angiotensin-(1-7) or a functional part, derivative and/or analogue thereof.

2. The method according to claim 1, wherein the cells comprise at least cells that, under normal circumstances, are not in direct contact with blood.

3. The method according to claim 2, wherein the cells are muscle cells.

4. The method according to claim 3, wherein the muscle cells are cardiac or skeletal muscle cells.

5. The method according to claim 3, wherein the cells are smooth muscle cells in the heart of an individual suffering from, or at risk of suffering from, heart pressure overload and/or myocardial infarction.

6. The method according to claim 1, wherein the delivery is vehicle comprising a nucleic acid delivery vehicle and the means for releasing angiotensin-(1-7) or a functional part, derivative and/or analogue thereof allows the release of a nucleic acid comprising at least one sequence encoding angiotensin-(1-7) or a functional part, derivative and/or analogue thereof, and which delivery vehicle further comprises a nucleic acid delivery carrier.

7. A method according to claim 6, wherein the nucleic acid delivery vehicle further comprises at least one sequence encoding an additional angiogenesis promoting factor.

8. The method according to claim 7, wherein said additional angiogenesis promoting-factor is VEGF, bFGF, angiopoietin-1, a nucleic acid encoding a protein capable of promoting nitric oxide production, or functional analogues or derivatives thereof.

9. The method according to claim 6, wherein the expression of at least one sequence is regulated by a signal.

10. The method according to claim 9, wherein said signal is provided by oxygen tension.

11. The method according to claim 6, wherein said nucleic acid delivery carrier is selected from the group consisting of a liposome, a virus particle, or a functional analogue or derivative of either thereof.

12. The method according to claim 7, wherein said nucleic acid delivery carrier comprises a Semliki Forest virus vector, an adenovirus vector or an adeno-associated virus vector.

13. The method according to claim 1, wherein the delivery vehicle comprises an implantable device.

14. The method according to claim 13, wherein the means for releasing angiotensin-(1-7) or a functional part, derivative and/or analogue thereof comprises a layer coated on the implantable device, which layer comprises angiotensin-(1-7) or a functional part, derivative and/or analogue thereof.

15. The method according to claim 13, wherein the implantable device comprises a stent.

16. A method of preventing and/or reducing neointima formation in an individual, said method comprising:
using angiotensin-(1-7) or a functional part, derivative and/or analogue thereof to prevent and/or reduce the formation of neointima.

17. A pharmaceutical preparation for preventing and/or reducing the formation of neointima in an individual, said pharmaceutical preparation comprising:
angiotensin-(1-7) or a functional part, derivative and/or analogue thereof presented in a pharmaceutically acceptable manner.

18. A delivery vehicle for preventing and/or reducing the formation of neointima, wherein the delivery vehicle comprising
an implantable device,
means for releasing an angiotensin-(1-7)or a functional part, derivative and/or analogue thereof to a subject associated with the device.

19. The delivery vehicle of claim 18, wherein the means for releasing an angiotensin-(1-7)or a functional part, derivative and/or analogue thereof comprises a layer which has been coated on the implantable device, which layer comprises an angiotensin-(1-7) or a functional part, derivative and/or analogue thereof.

20. A pharmaceutical preparation for preventing and/or reducing the formation of neointima, said pharmaceutical preparation comprising:
the delivery vehicle of claim 18 presented in a pharmaceutically acceptable manner.

21. A method for preventing and/or reducing vascular wall hypertrophy, said method comprising:
delivering to cells of an individual, via a delivery vehicle comprising means for releasing angiotensin-(1-7) or a functional part, derivative and/or analogue thereof, angiotensin-(1-7) or a functional part, derivative and/or analogue thereof.
